(19) United States
(12) Patent Application Publication
(10) Pub. No.: US 2021/0353650 A1
(43) Pub. Date: Nov. 18, 2021

(54) PHARMACEUTICAL FORMULATION CONTAINING REMDESIVIR AND ITS ACTIVE METABOLITES FOR DRY POWDER INHALATION

(71) Applicants: Cai Gu Huang, Shrewsbury, MA (US); Abid Hussain, Shanghai (CN)

(72) Inventors: Cai Gu Huang, Shrewsbury, MA (US); Abid Hussain, Shanghai (CN)

(73) Assignee: Cai Gu Huang, Shrewsbury, MA (US)

(21) Appl. No.: 17/323,109

(22) Filed: May 18, 2021

Related U.S. Application Data
(60) Provisional application No. 63/042,977, filed on Jun. 23, 2020.
(60) Provisional application No. 63/026,663, filed on May 18, 2020.

(51) Int. Cl. A61K 31/675; A61K 9/00; A61K 47/06; A61K 9/16

(52) U.S. Cl. CPC A61K 31/675; A61K 9/1623; A61K 47/06; A61K 9/0075

(57) ABSTRACT

The invention provides a pharmaceutical composition for dry powder inhalation and a preparation method thereof, wherein the composition comprises, a carrier material, preferably lactose and micronized remdesivir and/or its active metabolites (such as Alanine metabolite (Ala-met), Nucleoside monophosphate, and Nucleoside Triphosphate (NTP)) and/or its analog GS-441524 and pharmaceutically acceptable salts thereof. The active pharmaceutical ingredient may be an anti-viral, taken as remdesivir and/or its active metabolites (such as Alanine metabolite (Ala-met), Nucleoside monophosphate. and Nucleoside Triphosphate (NTP)) and/or its analog GS-441524. The dry powder inhalation containing Remdesivir and/or its active metabolites and/or its analog GS-441524 as active ingredients, further consisting of a breath-powered, dry powder inhaler, and a cartridge for delivering a dry powder formulation deep into the lungs for the treatment of respiratory disorders. The inhaler and cartridge can be provided with a drug delivery formulation comprising, for example, an active ingredient, including, small organic molecules, including, remdesivir and/or its active metabolites (such as Alanine metabolite (Ala-met), Nucleoside monophosphate, and Nucleoside Triphosphate (NTP)) and/or its analog GS-441524 and pharmaceutically acceptable salts thereof for the treatment of disease and disorders, for example, COVID-19 and other viral respiratory infections.

### PRIORITY STATEMENT

This application claims the benefit of the filing date of U.S. Provisional Patent Application No. 63/026,663, filed on May 18, 2020, and Application No. 63/042,977, filed on Jun. 23, 2020, which are incorporated herein by reference in their entirety.

### BACKGROUND OF THE INVENTION

Viral infections are frequently highly contagious, especially when spread by respiration. The recent COVID-19 pandemic, now known to be caused by a corona virus, shows how rapidly an infection can spread when it is transmitted through air contact. Other diseases such as influenza also spread by air contact, and can rapidly reach epidemic proportions, with high numbers of fatalities in elderly and immune-compromised populations.

The primary way that SARS-CoV appears to spread is by close person-to-person contact. Most cases of SARS-CoV have involved people who cared for, or lived with, someone with SARS-CoV, or had direct contact with infectious material (for example, respiratory secretions) from a person who has SARS-CoV. A potential way in which SARS-CoV can be spread involves touching the skin of other people or objects that are contaminated with infectious droplets and then touching your eye(s), nose, or mouth. Spread can also happen when someone who is sick with SARS-CoV coughs or sneezes droplets onto themselves, other people, or nearby surfaces. It also is possible that SARS-CoV can be spread through the air or by ways that are currently not known. At present there is no treatment for or means of preventing SARS-CoV, other than supportive care. However, in some countries there have been announcements of promising outcomes with remdesivir administration.

Remdesivir, chemically 2-ethylbutyl((S)-(((2R,3S,4R,5R)-5-(4-aminopyrrolo[1,2-b]pyridazin-7-yl)-5-cyano-3,4-dihydroxytetrahydrofuran-2-yl)methoxy)(phenoxy)phosphoryl)-L-alaninate, has the following chemical structure:

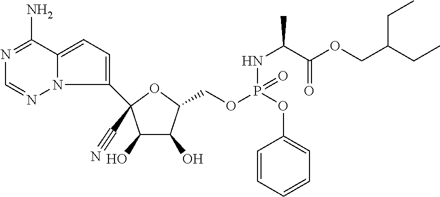 [SMILES: CCC(CC)COC(=O)[C@H](C)NP(=O)(OC[C@H]1O[C@@](C#N)(c2ccc3c(N)ncnn23)[C@H](O)[C@@H]1O)Oc1ccccc1]

The parent drug remdesivir hydrolyzes to its active metabolites such as Alanine metabolite (Ala-met), nucleoside monophosphate, and finally to nucleoside triphosphate (NTP). The chemical structures of each metabolite is given below:

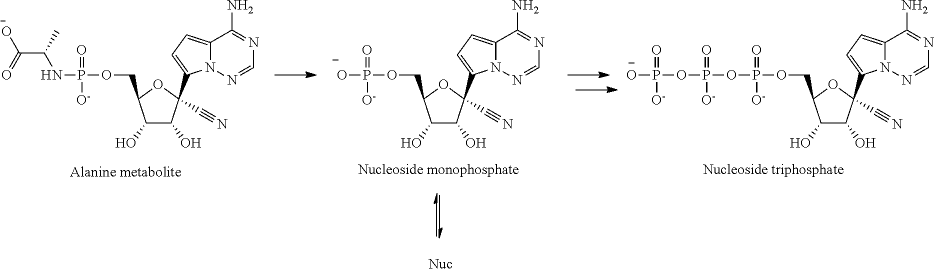 [SMILES: C[C@H](NP(=O)([O-])OC[C@H]1O[C@@](C#N)(c2ccc3c(N)ncnn23)[C@H](O)[C@@H]1O)C(=O)[O-] ; N#C[C@@]1(c2ccc3c(N)ncnn23)O[C@H](COP(=O)([O-])[O-])[C@@H](O)[C@H]1O ; N#C[C@@]1(c2ccc3c(N)ncnn23)O[C@H](COP(=O)([O-])OP(=O)([O-])OP(=O)([O-])[O-])[C@@H](O)[C@H]1O]

Remdesivir has been found to show antiviral activity against viruses such as respiratory syncytial virus, Junin virus, Lassa fever virus, and Coronavirus. Remdesivir was rapidly pushed through clinical trials due to the West African Ebola virus epidemic crisis.

Remdesivir was active against a broad spectrum of viral pathogens, which included Middle East Respiratory Syndrome (MERS) virus, severe acute respiratory syndrome CoV (SARS-CoV), Marburg virus, and multiple variants of Ebola virus, including the Makona strain, which caused the most recent outbreak in Western Africa. Recent studies showed remdesivir may be effective against the new coronavirus 2019-nCoV emerging worldwide.

A 1-cyano-substituted adenine C-nucleoside ribose analogue (Nuc) exhibits antiviral activity against a number of RNA viruses. The mechanism of action of Nuc requires intracellular anabolism to the active triphosphate metabolite (NTP), which is expected to interfere with the activity of viral RNA-dependent RNA-polymerases (RdRp). Structurally, the 1-cyano group provides potency and selectivity towards viral RNA polymerases, but because of slow first phosphorylation kinetics, modification of the parent nucleoside with monophosphate promoieties has the potential to greatly enhance intracellular NTP concentrations. The parent drug is a single Sp isomer of the 2-ethylbutyl 1-alaninate phosphoramidate prodrug that effectively bypasses the rate-limiting first phosphorylation step of the Nuc.

Remdesivir is a pro-drug of its parent adenosine analog, which is metabolized into an active nucleoside triphosphate (NTP) by the host, and currently is an investigational broad-spectrum small-molecule antiviral drug that has demonstrated activity against RNA viruses in several families, including Coronaviridae (such as SARSCoV, MERS-CoV, and strains of bat coronaviruses capable of infecting human respiratory epithelial cells), Paramyxoviridae (such as Nipah virus, respiratory syncytial virus, and Hendra virus), and Filoviridae (such as Ebola virus). Remdesivir was originally developed to treat Ebola virus infections.

As a nucleoside analog, remdesivir acts to interfere with RNA-dependent RNA polymerase, targeting the viral genome replication process. The RNA-dependent RNA polymerase is the protein complex CoVs uses to replicate their RNA-based genomes. After the host metabolizes remdesivir into the active nucleoside triphosphate, the metabolite competes with adenosine triphosphate for incorporation into the nascent RNA strand. The incorporation of this substitute into the new strand results in premature termination of RNA synthesis, halting the growth of the RNA strand after a few more nucleotides are added. Although CoVs have a proof-reading process that is able to detect and remove other nucleoside analogs, rendering them resistant to many of these drugs, the active metabolites of remdesivir seem to outpace this viral proof-reading activity, thus maintaining antiviral activity.

Another one of remdesivir's analogs is GS-441524, and its chemical structure is given below:

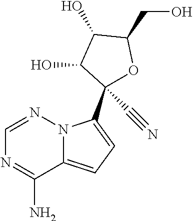 [SMILES: N#C[C@@]1(c2ccc3c(N)ncnn23)O[C@H](CO)[C@@H](O)[C@H]1O]

GS-441524 has certain antiviral activity against hepatitis C virus, dengue virus, pandemic influenza virus, parainfluenza virus and SARS coronavirus, and has achieved good results in experiments on cats.

Remdesivir is usually administered intravenously, due to difficulties in administering it as an injectable solution. There are, however, side effects associated with intravenous administration due to the long infusion time. An inhalation route is a preferred administration route for the delivery of drugs for the treatment of most respiratory diseases.

Surprisingly, we have found a new delivery method that more effectively and selectively delivers remdesivir and/or its active metabolites and/or its analog (GS-441524). This method advantageously improves deposition of remdesivir metabolites in the lungs so that it can more effectively inhibit and remove the virus from lung and other parts of human body. This new delivery method involves dry powder inhalation and presents clear and significant clinical benefits, such as improved availability at the target site, higher efficacy, and less side effects.

Furthermore, the delivery method, which involves administration by inhalation, is advantageous in that it can achieve a better distribution of remdesivir and/or its active metabolites and/or its analog GS-441524 in the lungs, which is beneficial when treating or curing a respiratory illness. Increased lung deposition of a drug delivered as dry powder inhalation is important.

### SUMMARY OF THE INVENTION

The present invention is in the field of pulmonary delivery of remdesivir and/or its active metabolites, such as Alanine metabolite (Ala-met), Nucleoside monophosphate, and Nucleoside Triphosphate (NTP), and/or remdesivir's analog GS-441524, and pharmaceutically acceptable salts and solvates thereof, to decrease or remove the viral load or accumulation of airborne pathogens inside the lungs or respiratory organs.

The present invention relates to powdered pharmaceutical formulations of remdesivir and/or its active metabolites, and/or its analog GS-441524, and pharmaceutically acceptable salts or solvates thereof, which can be administered by dry powder inhalation, using lactose as a carrier material. The powdered pharmaceutical formulations according to the invention meet high quality standards.

One aspect of the present invention is to provide a pharmaceutical formulation containing remdesivir and/or its active metabolites and/or its analog GS-441524 that meets the high standards required for dry powder inhalation. The stability of the active substances in the formulation should be a storage time of some years. In one embodiment, the stability of the active substances in the formulation is more one year. In one embodiment, the stability of the active substances in the formulation is more than three years.

Another aspect of the invention is to provide formulations of solutions containing remdesivir and/or its active metabolites and/or its analog GS-441524 that is inhaled under pressure using an inhaler, the composition is delivered as a dry powder or aerosol having a particle size falling reproducibly within a specified range.

Another aspect of the invention is to provide a dry powder formulation comprising remdesivir and/or its active metabolites and/or its analog GS-441524 and other inactive excipients, such as lactose, which can be administered as dry powder. In one embodiment, the active ingredient such as, remdesivir and/or its active metabolites and/or its analog GS-441524, has a mass median aerodynamic diameter ranging from about 1 micron to about 5 microns. This particle size is able to penetrate the lung on inhalation.

An aspect of the current invention is to provide a more effective and easy to administer dry powder inhalation dosage form containing anti-viral active ingredients, such as, remdesivir and/or its active metabolites and/or its analog GS-441524, such as Alanine metabolite (Ala-met), Nucleoside monophosphate, Nucleoside Triphosphate (NTP). and GS-441524, and pharmaceutically acceptable salts and solvates thereof, using a carrier material, such as lactose, for the treatment of respiratory infections caused by SARS-CoV.

Another aspect is to provide a dry powder formulation, which has substantial long term stability. In one embodiment, the formulations can be stored at a temperature of from about 1° C. to about 30° C.

A further aspect of the invention is to provide a method or process to prepare the dry powder inhalation formulation of remdesivir and/or its active metabolites and/or its analog GS-441524.

These and other features, aspects and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

FIG. 1 depicts illustrative multi-dose dry powder inhalers.

### DETAILED DESCRIPTION OF THE INVENTION

Using an inhalation formulation to administer an active substance achieves a better distribution of the active substance in the lungs. It is important to increase the lung deposition of an active substance being delivered by inhalation.

Dry powder inhalers (DPI) are known and are used to treat respiratory diseases by delivering a dry powder comprising an active substance in aerosol form through inhalation to the patients' airways. For delivery deep into the lungs, particles in the range of about 1 micron to about 5 microns are required. In pharmaceutical dry powders, the active pharmaceutical ingredient (API) is agglomerated on the surface of larger carrier particles, such as, but not limited to, lactose. The DPI's therefore operate by complex mechanisms to ensure that such agglomerates disperse and break up, or disaggregate, before the API can be inhaled deep into the lungs. Pharmaceutical dry powders containing lactose as a carrier are typically in the range of about 20 microns to about 100 microns. Existing DPI's, typically first “grind” or de-agglomerate the dry powder or impact the larger particles of the dry powder to result in the aforementioned particle size range.

DPI's rely on the force of the patient's inhalation to extract the powder from the device and to break-up the powder into particles that are small enough to enter the lungs. Sufficiently high inhalation rates are required to ascertain correct dosing and complete disaggregation of the powder. Typically, a large amount of API remains attached to the surface of the carrier and is deposited in the upper airways due to incomplete de-aggregation of the powder. Inhalation rates of existing DPI's are usually in the range of about 40 to about 120 liters/min (L/min).

It is desirable to have a remdesivir powder inhaler that can deliver remdesivir powder to a user at an inhalation or air flow rate that is suitable for treating COVID-19 and other viral infections that targets the respiratory systems and can cause damage to respiratory organs.

Prophylactic administration of a formulation containing one or more materials that alter the physical properties, such as surface tension and surface elasticity, of mucus lining fluid of the lung can be used to help reduce viral shedding and spread of bacterial infection.

Lung mucociliary clearance is the primary mechanism by which the airways are kept clean from particles. The particles are present in a liquid film that coats airways. The conducting airways are lined with ciliated epithelium that beat to drive a layer of mucus, containing the particles, towards the larynx, clearing the airways from the lowest ciliated region in about 24 hours. The fluid coating the airways consists of water, sugars, proteins, glycoproteins, and lipids. It is generated in the airway epithelium and the submucosal glands, and the thickness of the layer ranges from several microns in the trachea to approximately 1 micron in the distal airways in humans, rats, and guinea pigs.

A second important mechanism for keeping the lungs clean is via momentum transfer from air flowing through the lungs across the mucus coating. Coughing increases this momentum transfer and is used by the body to aid in the removal of excess mucus. It becomes important when mucus cannot be adequately removed by ciliary beating alone, as occurs with mucus hypersecretion that is associated with many disease states. Air speeds as high as 200 m/s can be generated during a forceful cough. For such high air speeds the onset of unstable sinusoidal disturbances at the mucus layer have been observed. This disturbance results in enhanced momentum transfer from the air to the mucus that accelerates the rate of mucus clearance from the lungs.

Formulations have been developed to limit infections of the respiratory system, especially viral infections of the lung. These formulations include a material which significantly alters the physical properties, such as surface tension and surface elasticity, of the lung mucus lining fluid as the principle active ingredient, carrier materials, and optionally, anti-viral such as, remdesivir and/or its active metabolites (such as Alanine metabolite (Ala-met), Nucleoside monophosphate, and Nucleoside Triphosphate (NTP)) and/or remdesivir's analog GS-441524 and pharmaceutically acceptable salts thereof. In one embodiment, the formulations are an organic suspension for enhanced delivery to the lung, that forms liquid aerosol particles having a diameter ranging from about 3 μm to about 7 μm that are loaded with a high concentration of an active substance such as a protein, surfactant, and/or biopolymer, which reduce viral shedding.

The geometry of the airways is a major barrier for drug dispersal within the lungs. The lungs are designed to entrap particles of foreign matter that are breathed in, such as dust. There are three basic mechanisms of deposition: impaction, sedimentation, and Brownian motion (J. M. Padfield. 1987. In: D. Ganderton & T. Jones eds. Drug Delivery to the Respiratory Tract, Ellis Harwood, Chicherster, U.K.). Impaction occurs when particles are unable to stay within the air stream, particularly at airway branches. The particles are adsorbed onto the mucus layer covering the bronchial walls and cleaned out by mucocilliary action. Impaction occurs mostly with particles over 5 μm in diameter. Smaller particles (<5 μm) can stay within the airstream and be transported deep into the lungs. Sedimentation often occurs in the lower respiratory system where airflow is slower. Very small particles (<0.6 μm) can be deposited by Brownian motion. This regime is undesirable because deposition cannot be targeted to the alveoli (see N. Worakul & J. R. Robinson. 2002. In: Polymeric Biomaterials, 2^{nd} ed. S. Dumitriu ed. Marcel Dekker. New York).

Another consideration when designing particles for aerosol delivery is the surface to volume ratio, which contributes to the efficiency of deposition. Particles with a large size and a low mass have proven most effective at deep lung deposition. This quality can be characterized by the aerodynamic diameter. The optimum aerodynamic diameter of the particles to achieve 60% deposition of the inhaled particles has to be approximately 3 μm. In one embodiment of the invention, the particle size ranges from about 3 microns to about 7 microns in diameter. However, particles up to about 15 microns can be utilized.

Drug delivery by inhalation represents a well-established mode of administration of low molecular weight pharmaceuticals to treat various lung disorders, by noninvasive systemic delivery of the pharmaceutical. Several biopharmaceutical companies are developing methods for pulmonary delivery of peptides and proteins, with one such product already in clinical use (the enzyme DNAse produced by Genentech for the treatment of symptoms of cystic fibrosis in children). Importantly, there is no evidence that inhaling autologous proteins presents significant immune issues.

The effective dose of an active pharmaceutical ingredient (such as remdesivir and/or its active metabolites) against COVID-19 depends on its bioavailability and clinical efficacy. In one embodiment, the effective dose of the active pharmaceutical ingredient against COVID-19 is between about 5 mg and about 500 mg. In one embodiment, the effective dose of the active pharmaceutical ingredient against COVID-19 is between about 10 mg and about 300 mg. In one embodiment, the effective dose of the active pharmaceutical ingredient against COVID-19 is between about 20 mg and about 100 mg.

A number of pharmaceutical preparations for pulmonary delivery of drugs have been developed. For example, U.S. Pat. No. 5,230,884 to Evans et al., discloses the use of reverse micelles for pulmonary delivery of proteins and peptides. Reverse micelles are formed by adding a small amount of water to a nonpolar solvent (e.g., hexane) to form micro-droplets. In this medium, a surfactant (detergent) orients itself with its polar heads inward, so that they are in contact with the water and the hydrophobic tails outward. The tiny droplets of water are surrounded by surfactant molecules, and the protein to be delivered is dissolved in the aqueous phase. U.S. Pat. No. 5,654,007 to Johnson et al., discloses methods for making an agglomerate composition containing a medicament powder (e.g., protein, nucleic acid, peptide, etc.) wherein a nonaqueous solvent binding liquid (a fluorocarbon) is used to bind the fine particles into aggregated units. The agglomerate composition has a mean size ranging from 50 to 600 microns and is allegedly useful for pulmonary drug delivery by inhalation.

PCT/US97/08895 by the Massachusetts Institute of Technology discloses particles made of a biodegradable material or drug, which have a tap density less than 0.4 g/cm and a mean diameter between 5 um and 30 um.

PCT/EP97/01560 by Glaxo Group Limited discloses spherical hollow drug particulates for use in pulmonary delivery. These materials are useful for delivering formulation to the lungs, and can be modified to deliver the correct dosage of a surface modifying agent at a desired rate and to a preferred location within the lung.

Dry powder formulations (“DPFs”) with large particle size have improved flowability characteristics, such as less aggregation (Visser, J., Powder Technology 58: 1-10 (1989)), easier aerosolization, and potentially less phagocytosis. Rudt, S. and R. H. Muller, J. Controlled Release, 22: 263-272 (1992); Tabata, Y., and Y. Ikada, J. Biomed. Mater. Res., 22: 837-858 40 (1988). Dry powder aerosols for inhalation therapy are generally produced with mean diameters in the range of less than 5 microns (see Ganderton, D., J. Biopharmaceutical Sciences, 3:101-105 (1992); and Gonda, I. “Physico-Chemical Principles in Aerosol Delivery,” in Topics in Pharmaceutical Sciences 1991; Crommelin, D. J. and K. K. Midha, Eds., Medpharm Scientific Publishers, Stuttgart, pp. 95-115, 1992), although a preferred aerodynamic diameter range is between one and ten microns. Large carrier particles (containing no drug) have been co-delivered with therapeutic aerosols to aid in achieving efficient aerosolization among other possible benefits. French, D. L., Edwards, D. A. and Niven, R. W., J. Aerosol Sci., 27: 769-783 (1996).

As noted above, particles can be formed solely of a surface modifying agent, or can be combined with a drug or excipient. Suitable materials for forming particles include proteins, such as albumin; polysaccharides, such as dextran; sugars, such as lactose; and synthetic polymers. Preferred polymers are biodegradable. In one embodiment, the polymer is a polyethyleneoxide copolymer, which has surfactant properties. In one embodiment, materials other than biodegradable polymers are used to form the particles, which can include other polymers and excipients. Other materials useful to form the particles include, but are not limited to, gelatin, polyethylene glycol, polyethylene oxide, trehalose, and dextran. Particles with degradation and release times ranging from seconds to months can be designed and fabricated, by established methods in the art.

The present invention further provides a pharmaceutical product comprising a compound, such as remdesivir and/or its active metabolites, wherein each compound is formulated with a pharmaceutically acceptable carrier or excipient. In one embodiment, the pharmaceutically acceptable carrier or excipient is lactose. In one embodiment, the compositions of the invention are suitable for inhalation, including fine particle powders, or mists, that can be generated and administered by means of various types of inhalers, such as, but not limited to, reservoir dry powder inhalers, unit-dose dry powder inhalers, pre-metered multi-dose dry powder inhalers, nasal inhalers, pressurized metered dose inhalers, and nebulizers or insufflators.

The compositions may be prepared by any of the methods well known in the pharmaceutical art s. In general, the methods for preparing the compositions include the steps of bringing the active ingredient(s), such as remdesivir and/or its active metabolites, such as Alanine metabolite (Ala-met), Nucleoside monophosphate, and Nucleoside Triphosphate (NTP), and/or remdesivir's analog GS-441524 and pharmaceutically acceptable salts and solvates thereof, into association with the carrier which may further include one or more accessory ingredients. In general, the compositions are prepared by uniformly and intimately bringing into association remdesivir and/or its active metabolites (such as Alanine metabolite (Ala-met), Nucleoside monophosphate, and Nucleoside Triphosphate (NTP)) and/or remdesivir's analog GS-441524 and pharmaceutically acceptable salts thereof, with liquid carriers or finely divided solid carriers, or both, and then, if necessary, shaping the product into the desired composition.

In one embodiment, the formulation of remdesivir and/or its active metabolites for administration by inhalation has a controlled particle size. In one embodiment, the particle size for inhalation into the bronchial system ranges from about 1 μm to about 10 μm. In one embodiment, the particle size for inhalation into the bronchial system ranges from about 2 μm to about 5 μm. Particles having a size above about 20 μm are generally too large when inhaled to reach the small airways. To achieve the desired particle size range the particles of remdesivir and/or its active metabolites and/or remdesivir's analog GS-441524 are reduced by conventional means including, but not limited to, micronization. In one embodiment, the fraction having the desired particle size is separated out by air classification or sieving. In one embodiment, the particles are crystalline.

In one embodiment, the powder composition contains a powder mix of the remdesivir and/or its active metabolites (such as Alanine metabolite (Ala-met), Nucleoside monophosphate, and Nucleoside Triphosphate (NTP)) and/or remdesivir's analog GS-441524 and pharmaceutically acceptable salts thereof and a suitable powder base (carrier/diluent/excipient substance). In one embodiment, the powder base is a mono-, di-, or poly-saccharide (e.g., lactose). In one embodiment, the powder base is lactose. In one embodiment, the lactose is selected from the group consisting of anhydrous lactose and α-lactose monohydrate. In one embodiment, the carrier is α-lactose monohydrate. In one embodiment, the dry powder compositions include, in addition to the remdesivir and/or its active metabolites, an additional excipient, such as, but not limited to, a sugar ester, calcium stearate, and magnesium stearate. In one embodiment, the particle size of the inactive ingredient ranges from about 10 micron to about 100 microns. In one embodiment, the effective dose of the active ingredients ranges from about 10 mg to about 150 mg. In one embodiment, the effective dose of the active ingredients ranges from about 10 mg to about 100 mg.

In one embodiment, the dry powder compositions according to the invention comprises a carrier. In one embodiment, the carrier is lactose in an amount ranging from about 30% to about 95% by weight of the formulation. In one embodiment, the carrier is lactose in an amount ranging from about 50% to about 80% by weight of the formulation. In one embodiment, the carrier is lactose in an amount ranging from about 60% to about 90% by weight of the formulation. In one embodiment, the carrier is α-lactose monohydrate in an amount ranging from about 30% to about 99% by weight of the formulation. In one embodiment, the carrier is α-lactose monohydrate in an amount ranging from about 50% to about 99.0% by weight of the formulation. In one embodiment, the carrier is α-lactose monohydrate in an amount ranging from about 60.0% to about 90% by weight of the formulation. In general, the particle size of the carrier, for example lactose, is greater than the inhaled active agent. In one embodiment, the carrier is milled lactose, having a MMD (mass median diameter) ranging from about 20 μm to about 100 μm. In one embodiment, the lactose component comprises a fine lactose fraction. The phrase “fine lactose fraction,” as used herein, means the fraction of lactose having a particle size of less than about 10 μm. In one embodiment, the fine lactose fraction has a particle size of less than about 6 μm. In one embodiment, the fine lactose fraction has a particle size of less than about 5 μm. In one embodiment, the particle size of the fine lactose fraction is less than about 4.5 μm. In one embodiment, the fine lactose fraction comprises from about 2% to about 10% by weight of the total lactose component. In one embodiment, the fine lactose fraction comprises from about 3% to about 6% by weight of the total lactose component. In one embodiment, the fine lactose fraction comprises about 4.5% by weight of the total lactose component.

In one aspect, the present invention provides a pharmaceutical combination product comprising remdesivir and/or its active metabolites (such as Alanine metabolite (Ala-met), Nucleoside monophosphate, and Nucleoside Triphosphate (NTP)) and/or remdesivir's analog GS-441524 and pharmaceutically acceptable salts thereof, wherein the formulation includes a pharmaceutically acceptable carrier. In one embodiment, the pharmaceutically acceptable carrier is lactose.

In one embodiment, the pharmaceutical formulation is presented in a unit dosage form. In one embodiment, the dry powder compositions for topical delivery to the lungs by inhalation is presented in capsules or cartridges of, for example, gelatin, or in blisters of, for example, laminated aluminum foil, for use in an inhaler or insufflator.

In one embodiment, each capsule, cartridge, or blister contains about 12.5 mcg of remdesivir and/or its active metabolites (such as, Alanine metabolite (Ala-met), Nucleoside monophosphate, and Nucleoside Triphosphate (NTP)) and/or remdesivir's analog GS-441524. In one embodiment, each capsule, cartridge, or blister contains about 25.0 mcg of remdesivir or its active metabolites. In one embodiment, each capsule, cartridge, or blister contains about 50.0 mcg of remdesivir or its active metabolites. In one embodiment, each capsule, cartridge, or blister contains about 100.0 mcg of remdesivir or its active metabolites. In one embodiment, each capsule, cartridge, or blister contains about 150.0 mcg of remdesivir or its active metabolites. In one embodiment, the formulation is packaged so as to be suitable for unit dose delivery. In one embodiment, the formulation is packaged so as to be suitable for multi-dose delivery. As indicated above, the remdesivir and/or its active metabolites may be formulated independently or in admixture with a carrier or excipients. The formulation can be provided in combination with or without additional carriers and/or excipients.

In one embodiment, the formulation is incorporated into a plurality of sealed dose containers provided on a medicament pack that can be mounted inside a suitable inhalation device. The containers may be rupturable, peelable, or otherwise openable one-at-a-time so that a dose of the dry powder composition can be administered by inhalation through the mouthpiece of an inhalation device, as is known in the art. The medicament pack can take a number of different forms including, but not limited to, a disk-shape and an elongated strip. Representative inhalation devices useful to administer the formulations of the invention include, but are not limited to, the DISKHALER™ and DISKUS™ devices, marketed by GlaxoSmithKline. The DISKUS™ inhalation device is described in GB 2242134A.

There are two types of dry powder inhalers: single-dose dry powder inhalers and multi-dose dry powder inhalers. The single-dose dry powder inhaler includes capsules and an inhalation device, wherein the inhalation device generally includes one mouthpiece, one grille, one capsule chamber, one or two puncture needles, and one dust cap. Generally, the grille is located between the mouthpiece and the capsule chamber, so as to prevent the capsule and larger pieces of the capsule from entering the mouthpiece, and plays a role in dispersing the drug particles. The puncture needle is placed on one or two sides of the capsule chamber to puncture the capsule. When the air flow is generated by inhalation, the drug particles in the capsule enter the internal channel of the mouthpiece channel through the punctured pore and finally enter the respiratory system. Illustrative single-dose dry powder inhalers include, but are not limited to, Aerolizer®, Handihaler®, Breezhaler®, Rotahaler®, and Spinhaler®.

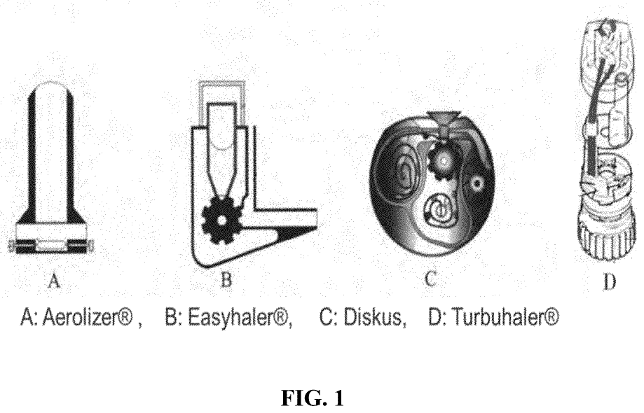

The multi-dose dry powder inhaler has a complicated structure, and a complicated air flow channel design and drug storage design. For example, a Turbuhaler® has 14 parts, including a medicine repository, a mouthpiece, and a counter. Illustrative multi-dose dry powder inhalers are depicted in FIG. 1 and include, but are not limited to, Turbuhaler®, Easyhaler®, Diskus, Novilizer®, and Diskhaler®, and the number of inhalations varies from 4 to 60. The Discus inhaler pre-separates each dose using a blister, and Diskhaler® separates each dose of drug powder using a capsule tray.

In one embodiment, the present invention provides remdesivir and/or its active metabolites and/or its analog GS-441524 in combination with an inhaler wherein the unit dose form is a capsule, cartridge or blister.

In one embodiment, the present invention provides remdesivir and/or its active metabolites and/or its analog GS-441524 in combination with an inhaler, wherein remdesivir and/or its active metabolites and/or its analog GS-441524 is present in an amount sufficient to deliver about 300.0 mcg/dose.

In one embodiment, the present invention provides remdesivir and/or its active metabolites and/or its analog GS-441524 in combination with an inhaler, wherein remdesivir and/or its active metabolites and/or its analog GS-441524 is present in an amount sufficient to deliver about 200.0 mcg/dose.

In a one embodiment, the present invention provides remdesivir and/or its active metabolites and/or its analog GS-441524 in combination with an inhaler, wherein remdesivir and/or its active metabolites and/or its analog GS-441524 is present in an amount sufficient to deliver about 100.0 mcg/dose.

In one embodiment, the present invention provides remdesivir and/or its active metabolites and/or its analog GS-441524 in combination with an inhaler, wherein remdesivir and/or its active metabolites and/or its analog GS-441524 is present in an amount sufficient to deliver about 50.0 mcg/dose.

In one embodiment, the present invention provides remdesivir and/or its active metabolites and/or its analog GS-441524 in combination with an inhaler, wherein remdesivir and/or its active metabolites and/or its analog GS-441524 is present in an amount sufficient to deliver about 25.0 mcg/dose.

In one embodiment, the present invention provides remdesivir and/or its active metabolites and/or its analog GS-441524 in combination with an inhaler, wherein remdesivir and/or its active metabolites and/or its analog GS-441524 is present in an amount sufficient to deliver about 12.5 mcg/dose.

In one embodiment, the present invention provides remdesivir and/or its active metabolites and/or its analog GS-441524 in combination with an inhaler, wherein remdesivir and/or its active metabolites and/or its analog GS-441524 is present in an amount sufficient to deliver about 7.5 mcg/dose.

In one embodiment, the present invention provides remdesivir and/or its active metabolites and/or its analog GS-441524 in combination with an inhaler, wherein remdesivir and/or its active metabolites and/or its analog GS-441524 is present in an amount sufficient to deliver about 2.5 mcg/dose.

In one embodiment, the compositions are formulated as a spray composition for inhalation. In one embodiment, the composition is formulated as an aqueous solution or suspension. In one embodiment, the composition is delivered as an aerosol from a pressurized pack, such as a metered dose inhaler (MDI), with the use of a suitable liquefied propellant. Aerosol compositions suitable for inhalation can be either a suspension or a solution and generally contain the active agent in combination with a suitable propellant. Illustrative propellants include, but are not limited to, a fluorocarbon, a hydrogen-containing chlorofluorocarbon, or mixtures thereof. Illustrative hydrofluoroalkanes include, but are not limited to, 1,1,1,2-tetrafluoroethane, 1,1,1,2,3,3,3-heptafluoro-n-propane, and mixtures thereof. The aerosol composition may optionally contain additional excipients well known in the art, such as surfactants (e.g., oleic acid, lecithin, or an oligolactic acid derivative such as as described in WO94/21229 and WO98/34596) and/or co-solvents (e.g., ethanol). Generally, the pressurized formulations are retained in a canister (e.g., an aluminum canister) closed with a valve (e.g. a metering valve) and fitted into an actuator provided with a mouthpiece.

One aspect of the invention is a pharmaceutical product comprising the remdesivir and/or its active metabolites and/or its analog GS-441524 in combination with a fluorocarbon or hydrogen-containing chlorofluorocarbon propellant, optionally in combination with a surface-active agent and/or a co-solvent. In one embodiment, the propellant is selected from 1,1,1,2-tetrafluoroethane, 1,1,1,2,3,3.3-heptafluoro-n-propane, and mixtures thereof.

One aspect of the invention is a pharmaceutical product consisting of remdesivir and/or its active metabolites and/or its analog GS-441524 in combination with a fluorocarbon or hydrogen-containing chlorofluorocarbon propellant, optionally in combination with a surface-active agent and/or a cosolvent. In one embodiment the propellant is selected from 1,1,1,2-tetrafluoroethane, 1,1,1,2,3,3,3-heptafluoro-n-propane, and mixtures thereof.

In one embodiment, the compositions of the invention are buffered by the addition of suitable buffering agents.

In one embodiment, the formulation is an intranasal sprays that is formulated with an aqueous or a non-aqueous vehicle in combination with one or more additional agents including, but not limited to, thickening agents; buffer salts or acid or alkali to adjust the pH; isotonicity adjusting agents; and anti-oxidants.

Clinical Study

As a starting point for discovery of remdesivir, a library of ˜1000 small molecules focused around nucleoside analogues was compiled, based on prior knowledge of effective antiviral compounds that target RNA viruses. Nucleosides are poorly cell-permeable (and therefore can have a low hit rate in cell-based screens, such as antiviral screens), so modified nucleosides such as monophosphate ester and phosphoramidate prodrugs composed a significant portion of the library. Such prodrugs are typically more permeable to cells and are then metabolized within the cells to liberate the nucleoside or phosphorylated nucleoside. While the data from the original full screen does not appear to have been disclosed, a 1′-CN modified adenosine C-nucleoside, along with a monophosphate prodrug, later renamed as remdesivir, was found to be highly potent. Remdesivir and its S-acyl-2-thioethyl monophosphate prodrug had previously been reported in 2012 as a potent lead from a series of 10-substituted 4-aza-7,9-dideazaadenosine C-nucleosides, with broad activity against a panel of RNA viruses, including yellow fever virus (YFV), Dengue virus type 2 (DENV-2), influenza A, para-influenza, and SARS. The primary assay used was the cytoprotection effect (CPE) assay, in which live virus is incubated with a target cell line, and the antiviral activity is inferred from the ability of a test agent to rescue cell death, measured using a standard cell viability reagent. The 2012 study, showed CPE activity against SARS strain Toronto 2 (IC₅₀=2.2 μM) without causing cytotoxicity toward the host Vero African green monkey kidney epithelial cells used in the CPE assay (note that different target cells were utilized in the viral CPE assays).

With the demonstration that remdesivir possessed broad activity against RNA viruses, multiple groups assessed antiviral activity both in vitro and in vivo, validating its activity against coronaviruses. Antiviral activity was confirmed against SARS, MERS zoonotic coronaviruses, as well as the circulating human coronaviruses HCoV-OC and HCoV-229E, causative agents of the common cold. Furthermore, de Wit et al. demonstrated that remdesivir and its active metabolites had both prophylactic and therapeutic activity against MERS in a non-human primate in vivo model.

The pharmacokinetics of remdesivir have been summarized in compassionate use documentation published by the European Medicines Agency (EMA, 2020). Remdesivir is administered via intravenous injection (IV) with a loading dose on day 1 (200 mg in adults, adjusted for body weight in pediatric patients) followed by a daily maintenance dose (100 mg in adults) for up to 10 days. In nonhuman primates, daily administration of 10 mg/kg of remdesivir yielded a short plasma half-life of the prodrug (t_{1/2}=0.39 h), but sustained intracellular levels of the triphosphate form.

In vitro and preclinical in vivo animal models supported the effectiveness of remdesivir against SARS-CoV-2 and related coronaviruses. These included a recent in vitro study of remdesivir assessing antiviral activity against SARS-CoV-2 (previously known as 2019-nCov, strain nCoV-2019BetaCoV/Wuhan/WIV04/2019) using qRT-PCR quantification of viral copy number in infected Vero E6 cells. This study demonstrated an IC₅₀ of 770 nM and an IC₉₀ equal to 1,760 nM (with cytotoxic concentration>100 mM). In addition, works by Sheahan et al. and de Wit et al. demonstrated in vivo efficacy of remdesivir at inhibiting viral replication and reducing viral related pathology against related coronaviruses. These findings, along with the safety profile of remdesivir in the clinical trial assessment against EBOV, support the evaluation of remdesivir and its active metabolites as a potential therapeutic drug for repurposing against the SARS-CoV-2 pandemic.

With the COVID-19 outbreak increasing in size and a lack of alternative therapeutics, two clinical trials using remdesivir were designed and initiated in China. On Feb. 5, 2020, a phase 3 randomized, quadruple-blind, placebo-controlled clinical trial was registered at Capital Medical University, with the goal to determine safety and efficacy of remdesivir in patients with mild to moderate SARS-CoV-2 infection (NCT04252664, since suspended). A day later, a second trial (NCT04257656, since terminated) was registered at the same location, focused on patients with advanced COVID-19 respiratory disease. Both trials had planned to track as the primary outcome the time to clinical improvement, up to 28 days: normalization of fever, oxygen saturation, and respiratory rate, and alleviation of cough which is sustained for 72 h. Both trials delivered remdesivir as a 200 mg loading dose on the first day, with 9 subsequent days of maintenance dosing at 100 mg; this regime is identical to that utilized in the previous NCT03719586 Ebola trial, which appears to be the model for all subsequent trials involving remdesivir.

### EXAMPLES

### Example 1

Pharmaceutical grade lactose monohydrate, complying with the requirements of Ph.Eur/USNF, was used. Before use, the lactose monohydrate was sieved through a coarse screen (for example, a screen with a mesh size of 500 microns or 800 microns). The level of fines in the lactose monohydrate, which can be measured by a Sympatec instrument, has about 4.5% w/w of the particles having a particle size less than 4.5 micron. Remdesivir and/or its active metabolites and/or its analog GS-441524 was micronised before use using an APTM microniser to give a mass median diameter of between about 1 micron and 5 microns. In one example, the mass median diameter is between about 2 microns and 5 microns

| Ingredient | Quantity |
|---|---|
| Remdesivir and/or its active | 40 mg |
| metabolites and/or its analog | |
| GS-441524 | |
| Milled Lactose (MMD of | 850 mg |
| 60-90 μm, with 4.5% w/w | |
| less than 4.5 micron) | |

### Example 2

Pharmaceutical grade lactose monohydrate, complying with the requirements of Ph.Eur/USNF, was used. Before use, the lactose monohydrate was sieved through a coarse screen (for example, a screen with a mesh size 500 microns or 800 microns). The level of fines in the lactose monohydrate, which can be measured by a Sympatec instrument, was about 4.5% w/w of the particles having a particle size of less than 4.5 micron. Remdesivir and/or its active metabolites and/or its analog GS-441524 was micronized before use using an APTM microniser to give a mass median diameter of between about 1 micron and about 5 microns. In one example, the mass median diameter is between about 2 microns and about 5 microns.

| Ingredient | Quantity |
|---|---|
| Remdesivir and/or its active | 50 mg |
| metabolites and/or its analog | |
| GS-441524 | |
| Milled Lactose (MMD of | 860 mg |
| 60-90 μm, with 4.5% w/w | |
| less than 4.5 micron) | |

### Example 3

Pharmaceutical grade lactose monohydrate, complying with the requirements of Ph.Eur/USNF, was used. Before use, the lactose monohydrate was sieved through a coarse screen (for example, a screen with a mesh size 500 microns or 800 microns). The level of fines in the lactose monohydrate, which can be measured by a Sympatec instrument, was about 4.5% w/w of the particles having a particle size less than 4.5 micron. Remdesivir and/or its active metabolites and/or its analog GS-441524 was micronised before use using an APTM microniser to give a mass median diameter of between about 1 micron and about 5 microns. In one example, the mass median diameter is between about 2 microns and about 5 microns.

| Ingredient | Quantity |
|---|---|
| remdesivir and/or its active | 30 mg |
| metabolites and/or its analog | |
| GS-441524 | |
| Milled Lactose (MMD of | 800 mg |
| 60-90 μm, with 4.5% w/w | |
| less than 4.5 micron) | |

### Example 4

Blister Strip Preparation is as follows:

The blended composition is transferred into blister strips (typical nominal mean quantity of blend per blister is about 12.5 mg to about 13.5 mg) of the type typically used for the supply of dry powder for inhalation. The blister strips were then sealed in the customary fashion. Powder blends of the active ingredients for blisters containing other quantities of the active substance, such as about 1 mg to about 100 mg or about 1 mg to about 50 mg per blister, can be prepared using the same procedure.

### Example 5

Dry Powder Inhalation Capsule Preparation is as follows:

The blended composition is transferred or filled into capsules (typical nominal mean quantity of blend per capsule is about 25 mg) of the type typically used for the supply of capsule based dry powder for inhalation. The capsules were then enclosed in the customary fashion. Powder blends of the active ingredients for capsules containing other quantities of the active substance, such as about 5 mg to about 100 mg or about 5 mg to about 50 mg per capsules, can be prepared using the same procedure.

### Example 6

Particle Size Distribution:

TABLE 1
Ingredient Contents of Sample 1 Inhalation Formulation

| Ingredient | Sample 1 |
|---|---|
| RV-MP | 30 mg |
| Milled Lactose | 800 mg |
| (MMD of 60-90 μm, | |
| with 4.5% w/w less | |
| than 4.5 micron) | |

TABLE 2
Ingredient Contents of Sample 2 Inhalation Formulation

| Ingredient | Sample 2 |
|---|---|
| RV-MP | 30 mg |
| Milled Lactose | 800 mg |
| (MMD of 60-90 μm, | |
| with 7% w/w less | |
| than 4.5 micron) | |

TABLE 3
Ingredient Contents of
Sample 3 Inhalation Formulation

| Ingredient | Sample 3 |
|---|---|
| RV-MP | 30 mg |
| Milled Lactose | 800 mg |
| (MMD of 60-90 μm, | |
| with 11% w/w less | |
| than 4.5 micron) | |

TABLE 4
Ingredient Contents of
Sample 4 Inhalation Formulation

| Ingredient | Sample 4 |
|---|---|
| RV-MP | 30 mg |
| Milled Lactose | 800 mg |
| (MMD of 60-90 μm, | |
| with 14% w/w less | |
| than 4.5 micron) | |

The Dry Powder Inhalation capsules of samples 1-4 are prepared by the same method as Example 5.

Particle Size Distribution:

The aerodynamic particle size distribution was determined using a Next Generation Impactor instrument (NGI).The inhaler used is powder aerosol device. The inhaler was held close to the NGI inlet until no aerosol was visible. The flow rate of the NGI was set to 90 L/minute and was operated under ambient temperature

Sample 1 was discharged into the NGI. Fractions of the dose were deposited at different stages of the NGI, in accordance with the particle size of the fraction. Each fraction was washed from the stage and analyzed using HPLC. The results are provided in Table 5 below.

TABLE 5
Single Dose Level Distribution and Aerodynamic Particle
Size Distribution of RV-MP Inhalation Formulation
Sample 1 Administered by Powder Aerosol Device

| RV-MP | Dosage | Percentage content | Cut-off |
|---|---|---|---|
| Deposited | (mcg) | at all levels | diameter (μm) |
| Capsule | 22.18 | 2.69% | / |
| Device | 162.27 | 19.71% | / |
| Pre-separator | 94.03 | 11.42% | / |
| Throat | 123.85 | 15.05% | / |
| Stage 1 | 12.64 | 1.54% | 6.48 |
| Stage 2 | 35.26 | 4.28% | 3.61 |
| Stage 3 | 87.87 | 10.68% | 2.3 |
| Stage 4 | 166.84 | 20.27% | 1.37 |
| Stage 5 | 85.20 | 10.35% | 0.76 |
| Stage 6 | 26.19 | 3.18% | 0.43 |
| Stage 7 | 5.94 | 0.72% | 0.26 |
| Micro-Orifice | 0.82 | 0.10% | 0 |
| Collector (MOC) | | | |

| | |
|---|---|
| Theoretical | 903.61 |
| dose (mcg) | |
| Actual test | 823.09 |
| dose (mcg) | |
| Recovery rate | 91.09% |
| Impactor Size | 408.12 mcg |
| Mass (ISM) | |
| Fine Particle | 49.58% |
| Fraction (FPF) | |

TABLE 6
Single Dose Level Distribution and Aerodynamic Particle
Size Distribution of RV-MP Inhalation Formulation
Sample 2 Administered by Powder Aerosol Device

| RV-MP | Dosage | Percentage content | Cut-off |
|---|---|---|---|
| Deposited | (mcg) | at all levels | diameter (μm) |
| Capsule | 20.60 | 2.46% | / |
| Device | 170.42 | 20.37% | / |
| Pre-separator | 90.80 | 10.85% | / |
| Throat | 153.28 | 18.32% | / |
| Stage 1 | 11.44 | 1.37% | 6.48 |
| Stage 2 | 29.45 | 3.52% | 3.61 |
| Stage 3 | 89.30 | 10.67% | 2.3 |
| Stage 4 | 158.67 | 18.97% | 1.37 |
| Stage 5 | 82.34 | 9.84% | 0.76 |
| Stage 6 | 24.80 | 2.96% | 0.43 |
| Stage 7 | 4.36 | 0.52% | 0.26 |
| Micro-Orifice | 1.14 | 0.14% | 0 |
| Collector (MOC) | | | |

| | |
|---|---|
| Theoretical dose (mcg) | 903.61 |
| Actual test dose (mcg) | 836.6 |
| Recovery rate % | 92.58% |
| Impactor Size | 390.06 mcg |
| Mass (ISM) | |
| Fine Particle | 46.62% |
| Fraction (FPF) | |

TABLE 7
Single Dose Level Distribution and Aerodynamic
Particle Size Distribution of RV-MP Inhalation
Formulation Sample 3 Administered by Powder Aerosol Device

| RV-MP | Dosage | Percentage content | Cut-off |
|---|---|---|---|
| Deposited | (mcg) | at all levels | diameter (μm) |
| Capsule | 26.74 | 3.26% | / |
| Device | 173.94 | 21.22% | / |
| Pre-separator | 103.38 | 12.61% | / |
| Throat | 160.13 | 19.53% | / |
| Stage 1 | 15.17 | 1.85% | 6.48 |
| Stage 2 | 31.26 | 3.81% | 3.61 |
| Stage 3 | 78.41 | 9.56% | 2.3 |
| Stage 4 | 123.50 | 15.06% | 1.37 |
| Stage 5 | 75.24 | 9.18% | 0.76 |
| Stage 6 | 25.02 | 3.05% | 0.43 |
| Stage 7 | 6.45 | 0.79% | 0.26 |
| Micro-Orifice | 0.63 | 0.08% | 0 |
| Collector (MOC) | | | |

| | |
|---|---|
| Theoretical dose (mcg) | 903.61 |
| Actual test dose (mcg) | 819.87 |
| Recovery rate % | 90.73% |
| Impactor Size | 340.51 mcg |
| Mass (ISM) | |
| Fine Particle | 41.53% |
| Fraction (FPF) | |

TABLE 8
Single Dose Level Distribution and Aerodynamic
Particle Size Distribution of RV-MP Inhalation
Formulation Sample 4 Administered by powder aerosol device

| RV-MP | Dosage | Percentage content | Cut-off |
|---|---|---|---|
| Deposited | (mcg) | at all levels | diameter (μm) |
| Capsule | 25.78 | 3.18% | / |
| Device | 183.04 | 22.61% | / |
| Pre-separator | 116.53 | 14.39% | / |
| Throat | 176.28 | 21.77% | / |
| Stage 1 | 10.85 | 1.34% | 6.48 |
| Stage 2 | 25.79 | 3.19% | 3.61 |
| Stage 3 | 73.54 | 9.08% | 2.3 |
| Stage 4 | 100.14 | 12.37% | 1.37 |
| Stage 5 | 73.36 | 9.06% | 0.76 |
| Stage 6 | 20.16 | 2.49% | 0.43 |
| Stage 7 | 3.61 | 0.45% | 0.26 |
| Micro-Orifice | 0.57 | 0.07% | 0 |
| Collector (MOC) | | | |

| | |
|---|---|
| Theoretical dose (mcg) | 903.61 |
| Actual test dose (mcg) | 809.65 |
| Recovery rate % | 89.60% |
| Impactor Size | 297.17 mcg |
| Mass (ISM) | |
| Fine Particle | 36.70% |
| Fraction (FPF) | |

The larger the FPF value, the higher the atomization efficiency.

It can be shown from Tables 6-8 that the DPI of the present invention have good lung deposition and have the best FPF when 4.5% w/w of the lactose has a mass median diameter of less than about 4.5% w/w.

1. A dry powder pharmaceutical formulation for administration by inhalation comprising:
(a) microparticles of an active substance selected from the group consisting of remdesivir, active metabolites of remdesivir, remdesivir's analog GS-441524, and combinations thereof; and
(b) microparticles of a pharmacologically acceptable carrier, wherein the microparticles of the active substance thereof have a mass median diameter of between about 1 μm and about 6 μm, and wherein the microparticles of the carrier have a mass median diameter of between about 10 μm and about100 μm.

2. The pharmaceutical formulation of claim 1, wherein the active substance is present in an amount of from about 1% to about 90% by weight of the formulation.

3. The pharmaceutical formulation of claim 1, wherein the pharmacologically acceptable carrier is lactose monohydrate.

4. The pharmaceutical formulation of claim 1, wherein the lactose has a fine lactose fraction with a mass median diameter of less than about 10 μm.

5. The pharmaceutical formulation of claim 1, further comprising a ternary agent.

6. The pharmaceutical formulation of claim 5, wherein the ternary agent is magnesium stearate.

7. The pharmaceutical formulation of claim 6, wherein the magnesium stearate is present in an amount of about 0.6% w/w of the pharmaceutical formulation.

8. A unit dose form comprising the pharmaceutical formulation of claim 1.

9. The unit dose form of claim 8, wherein the unit dose is selected from the group consisting of a capsule, a cartridge, and a blister pack.

10. The unit dose form of claim 8, wherein the active substance is present in an amount of about 1 mg/dose to about 300 mg/dose.

11. The unit dose form of claim 8, wherein the active substance is present in an amount of about 10 mg per dose to 100 mg/dose.

12. The unit dose form of claim 8, wherein the active substance is present in an amount of selected from about 12.5 mg/dose, about 25 mg/dose, and about 50 mg/dose.

13. (canceled)

14. (canceled)

15. The pharmaceutical formulation of claim 1, further comprising a surface active agent.

16. A device for administering an active substance selected from the group consisting of remdesivir, active metabolites of remdesivir, and combinations thereof comprising microparticles comprising the active substance and a pharmacologically acceptable carrier,
wherein the microparticles of remdesivir, active metabolites of remdesivir, and combinations thereof have a mass median diameter of between about 1 and about 10 μm, and
wherein the microparticles are contained in a medicament dispenser selected from the group consisting of a reservoir dry powder inhaler, a unit-dose dry powder inhaler, a pre-metered multi-dose dry powder inhaler, a nasal inhaler, and a pressurized metered dose inhaler.

17. A pharmaceutical formulation for administration by inhalation comprising:
(i) microparticles of an active substance selected from the group consisting of remdesivir, active metabolites of remdesivir and combinations thereof;
(ii) a pharmacologically acceptable carrier,
wherein the microparticles of the active substance have a mass median diameter of between about 1 and about 10 μm, and
(iii) a propellant
wherein the microparticles are suspended in the propellant.

18. The pharmaceutical formulation of claim 17, wherein the propellant is selected from the group consisting of a fluorocarbon and hydrogen-containing chlorofluorocarbon.

19. The pharmaceutical formulation of claim 18, wherein the propellant is a hydrofluoroalkane.

20. The pharmaceutical formulation of claim 18, wherein the propellant is selected from the group consisting of 1,1,1,2-tetrafluoroethane; 1,1,1,2,3,3,3-heptafluoro-n-propane; and mixtures thereof.

21. The pharmaceutical formulation of claim 17, further comprising a surface active agent.

22. The pharmaceutical formulation of claim 17, wherein the microparticles of the active substance have a mass median diameter of between about 3 and about 7 μm.

23. A method of treating a virus infection in a patient comprising administering to the patient the pharmaceutical formulation of claim 1 by oral inhalation or nasal inhalation.

24. The method of claim 23, wherein the active substance is administered at a daily dose ranging from about 10 mg to about 500 mg.

25. The method of claim 24, wherein the active substance is administered at a daily dose ranging from about 50 mg to about 300 mg.

26. The method of claim 23, wherein the virus is selected from the group consisting of Ebola and Marburg virus (Filoviridae); coronavirus, new coronavirus COVID-19, Ross River virus, chikungunya virus, Sindbis virus, eastern equine encephalitis virus (Togaviridae, Alphavirus), vesicular stomatitis virus (Rhabdoviridae, Vesiculovirus), Amapari virus, Pichindé virus, Tacaribe virus, Junin virus, Machupo virus (Arenaviridae, Mammarenavirus), West Nile virus, dengue virus, yellow fever virus (Flaviviridae, Flavivirus); human immunodeficiency virus type 1 (Retroviridae, Lentivirus); Moloney murine leukemia virus (Retroviridae, Gammaretrovirus); respiratory syncytial virus (Paramyxoviridae, Pneumovirinae, Pneumovirus); vaccinia virus (Poxviridae, Chordopoxvirinae, Orthopoxvirus); herpes simplex virus type 1, herpes simplex virus type 2 (Herpesviridae, Alphaherpesvirinae, Simplexvirus); human cytomegalovirus (Herpesviridae, Betaherpesvirinae, Cytomegalovirus); Autographa californica nucleopolyhedrovirus (Baculoviridae, Alphabaculoviridae) (an insect virus); Semliki Forest virus, O'nyong-nyong virus, rubella (German measles) virus (Togaviridae, Rubivirus); rabies virus, Lagos bat virus, Mokola virus (Rhabdoviridae, Lyssavirus); Guanarito virus, Sabia virus, Lassa virus (Arenaviridae, Mammarenavirus); Zika virus, Japanese encephalitis virus, St. Louis encephalitis virus, tick-borne encephalitis virus, Omsk hemorrhagic fever virus, Kyasanur Forest virus (Flaviviridae, Flavivirus); human hepatitis C virus (Flaviviridae, Hepacivirus); influenza A/B virus (Orthomyxoviridae, the common ‘flu’ virus); Hendra virus, Nipah virus (Paramyxoviridae, Paramyxovirinae, Henipavirus); measles virus (Paramyxoviridae, Paramyxovirinae, Morbillivirus); variola major (smallpox) virus (Poxviridae, Chordopoxvirinae, Orthopoxvirus); human hepatitis B virus (Hepadnaviridae, Orthohepadnavirus); Middle East Respiratory Syndrome (MERS) virus, severe acute respiratory syndrome CoV (SARS-CoV), Marburg virus, and hepatitis delta virus (hepatitis D virus).

27. A method of treating a virus infection in a patient comprising administering to the patient the pharmaceutical formulation of claim 17 by oral inhalation or nasal inhalation.

28. The method of claim 27, wherein the active substance is administered at a daily dose ranging from about 10 mg to about 500 mg.

29. The method of claim 28, wherein the active substance is administered at a daily dose ranging from about 50 mg to about 300 mg.

30. The method of claim 27, wherein the virus is selected from the group consisting of Ebola and Marburg virus (Filoviridae); coronavirus, new coronavirus COVID-19, Ross River virus, chikungunya virus, Sindbis virus, eastern equine encephalitis virus (Togaviridae, Alphavirus), vesicular stomatitis virus (Rhabdoviridae, Vesiculovirus), Amapari virus, Pichindé virus, Tacaribe virus, Junin virus, Machupo virus (Arenaviridae, Mammarenavirus), West Nile virus, dengue virus, yellow fever virus (Flaviviridae, Flavivirus); human immunodeficiency virus type 1 (Retroviridae, Lentivirus); Moloney murine leukemia virus (Retroviridae, Gammaretrovirus); respiratory syncytial virus (Paramyxoviridae, Pneumovirinae, Pneumovirus); vaccinia virus (Poxviridae, Chordopoxvirinae, Orthopoxvirus); herpes simplex virus type 1, herpes simplex virus type 2 (Herpesviridae, Alphaherpesvirinae, Simplexvirus); human cytomegalovirus (Herpesviridae, Betaherpesvirinae, Cytomegalovirus); Autographa californica nucleopolyhedrovirus (Baculoviridae, Alphabaculoviridae) (an insect virus); Semliki Forest virus, O'nyong-nyong virus, rubella (German measles) virus (Togaviridae, Rubivirus); rabies virus, Lagos bat virus, Mokola virus (Rhabdoviridae, Lyssavirus); Guanarito virus, Sabia virus, Lassa virus (Arenaviridae, Mammarenavirus); Zika virus, Japanese encephalitis virus, St. Louis encephalitis virus, tick-borne encephalitis virus, Omsk hemorrhagic fever virus, Kyasanur Forest virus (Flaviviridae, Flavivirus); human hepatitis C virus (Flaviviridae, Hepacivirus); influenza A/B virus (Orthomyxoviridae, the common ‘flu’ virus); Hendra virus, Nipah virus (Paramyxoviridae, Paramyxovirinae, Henipavirus); measles virus (Paramyxoviridae, Paramyxovirinae, Morbillivirus); variola major (smallpox) virus (Poxviridae, Chordopoxvirinae, Orthopoxvirus); human hepatitis B virus (Hepadnaviridae, Orthohepadnavirus); Middle East Respiratory Syndrome (MERS) virus, severe acute respiratory syndrome CoV (SARS-CoV), Marburg virus, and hepatitis delta virus (hepatitis D virus).

31. The pharmaceutical formulation of claim 1, wherein the active substance is present in an amount ranging from about 30 mg to about 50 mg, the pharmacologically acceptable carrier is lactose in an amount ranging from about 800 to about 860 mg, the lactose has a mass median diameter of between about 60 μm and 90 μm, and about 4.5% w/w of the lactose has a mass median diameter of less than about 4.5% w/w.
